(19) United States
(12) Patent Application Publication
(10) Pub. No.: US 2014/0193923 A1
(43) Pub. Date: Jul. 10, 2014

(54) SENSORS AND METHODS FOR DETECTING PEROXIDE BASED EXPLOSIVES

(71) Applicant: University of Utah Research Foundation, (US)

(72) Inventors: Ling Zang, Salt Lake City, UT (US); Jimin Han, Salt Lake City, UT (US); Miao Xu, Salt Lake City, UT (US)

(21) Appl. No.: 13/865,846

(22) Filed: Apr. 18, 2013

Related U.S. Application Data
(60) Provisional application No. 61/635,091, filed on Apr. 18, 2012.

(51) Int. Cl. G01N 33/22; G01N 31/22

(52) U.S. Cl. CPC G01N 33/227; G01N 31/228
USPC 436/135; 422/420

(57) ABSTRACT

Methods, compositions, and systems for detecting explosives is disclosed and described. A sensor for detecting explosives can comprise a porous hydrophilic material modified with a series of π conjugated molecules with the general structure:

[EW]-[CS]-[PED]

wherein EW is an electron withdrawing group, CS is a conjugated system, and where PED is a pre-electron donating group. The porous hydrophilic material is capable of detecting hydrogen peroxide by reacting the aryl boronate group (or other Pre-ED groups) with the hydrogen peroxide to provide a fluorescent change.

### RELATED APPLICATION

This application claims the benefit of U.S. Provisional Patent Application No. 61/635,091 filed Apr. 18, 2012, which is incorporated herein by reference.

### GOVERNMENT INTEREST

This invention was made with government support under Award No. 2009-ST-108-LR0005 awarded by the U.S. Department of Homeland Security. The Government has certain rights in the invention.

### BACKGROUND OF THE INVENTION

Explosive devices can be difficult to detect as they can contain a variety of materials. In particular, many explosive detection systems focus on conventional explosives and generally do not detect peroxide based explosives. A large portion of improvised explosive devices utilize relatively simple and inexpensive peroxide based explosives. Currently, there are several sensor systems or devices commercially available for peroxide explosives detection, including those built on chromatography, mass spectrometry and enzyme catalysis. Various methods of detection can include requiring a sample of the object material to be tested. Additionally, many are directed towards chemical identification, for which the integrated multistep instrumentation procedures are often time-consuming, taking minutes or even tens of minutes, and intrusive. Such methods and systems are not suited for expedient, onsite explosives screening or monitoring, particularly when moving individuals or vehicles are involved. For vapor based detection of hydrogen peroxide, canines are sometimes considered an effective method of explosives detection. However, as dogs fatigue, their performance declines with time and they suffer from mood and behavior variations. Ion mobility spectrometry (IMS) is a good alternative to canines; however, IMS is prone to false alarms and requires frequent recalibration. Mass spectroscopy (MS) is highly sensitive to a broad class of explosives, but needs sample preparation/transfer, vacuum operation and ionizability of targets. FLIR uses glowing-stick like chemiluminescence technology to achieve vapor sensing of hydrogen peroxide. However, the detection limit is largely bottle-necked by the solid thin film materials (surface coated) that limits both the surface area and air sampling. Detection of these explosives through direct sensing of the peroxide compounds remains difficult mainly due to the weak oxidizing power (weak electron affinity) and lack of nitro-groups, which prevent the detection through fluorescence sensing (usually based on electron transfer quenching) and the conventional electronic detection systems, respectively.

### BRIEF DESCRIPTION OF THE DRAWINGS

The present invention will become more fully apparent from the following description and appended claims, taken in conjunction with the accompanying drawings. Understanding that these drawings merely depict exemplary embodiments of the present invention and they are, therefore, not to be considered limiting of its scope. It will be readily appreciated that the components of the present invention, as generally described and illustrated in the figures herein, could be arranged, sized, and designed in a wide variety of different configurations. Nonetheless, the invention will be described and explained with additional specificity and detail through the use of the accompanying drawings in which:

FIG. 1 shows the illustration of the sensor molecular structure and sensing mechanism. EW=Electron Withdraw; ED=Electron Donating; Pre-ED represents a pre-electron donating group which can transition into (e.g. by group modification or replacement) an ED group by reacting with H₂O₂. The sensor molecules are non-fluorescent or weak fluorescent at pristine state, which means a ‘zero’ background, thus suitable for being used as ‘turn-on’ fluorescence sensors. Upon reaction with hydrogen peroxide, the sensor molecule forms an intramolecular charge transfer state by generating a strong intermolecular push-pull system through the π-conjugation linker, which not only increases the molar extinction coefficient in the visible light region (leading to colorimetric sensing), but also turn-on the fluorescence (enabling fluorescence sensing).

FIG. 2 shows non-limiting examples of electron withdrawing groups. The sensor molecule can contain one or more electron withdrawing EW groups.

FIG. 3 shows examples of pre-electron donating groups and their relevant electron donating forms to which they can transition following reaction with hydrogen peroxide. The sensor molecule can contain one or more pre-electron donating groups. R can be an organic compound containing alkyl groups, aryl groups, or mixtures thereof.

FIG. 4 shows examples of conjugation link groups. The sensor molecule can contain one or more conjugation linker groups.

FIG. 5 shows examples of hydrophilic side chains that can be incorporated into the sensor molecules of the present invention. In particular, the side chains can be attached to the electron withdrawing and/or the conjugated linker/system component of the system.

FIGS. 6A and 6B show examples of sensor molecules. The sensor molecule can be any combination of one or more pre-electron donating, electron withdrawing group, conjugation linker and side chain.

FIGS. 7A and 7B show the turn-on fluorescence sensor for trace detection of hydrogen peroxide. FIG. 7A shows a daylight photograph (showing the color change upon formation of the product) and emission image taken in dark over an ethanol solution of the fluorophore sensor molecule. Also shown is the fluorescence spectrum measured over the same solution before and after reaction with hydrogen peroxide. FIG. 7B shows an example of the reaction of the sensor molecule C6NIB with H₂O₂ under alkaline conditions resulting in the transition or conversion of the aryl boronate group to phenol, thus turning on the charge transfer band fluorescence centered around 550 nm (a). 7B further shows that the turn-on sensing of C6NIB is extremely selective for H₂O₂ as tested (b).

FIG. 8 shows an example synthetic route of sensory molecules in an embodiment of the invention.

FIG. 9 shows a reaction between aryl boronate group and hydrogen peroxide, showing the fluorescence turn-on mechanism of an embodiment of the invention.

FIG. 10 shows the absorption and fluorescence spectra of an ethanol solution of C6NIB (5×10⁻⁶ mol/L, in the presence of 5×10⁻⁴ mol/L TBAH) before (black) and after (red) addition of 5×10⁻³ mol/L H₂O₂. (c, d) Extinction (converted from reflection spectrum) and fluorescence spectra of C6NIB dispersed in a 1.5×1.5 cm² silica gel TLC plate (containing 0.5 μmol C6NIB and 5 μmol TBAH) before (black) and after (red) exposure to 225 ppm H₂O₂ vapor for 5 min.

FIG. 11 shows absorption and fluorescence spectra of C6NIB, C6NIO, and the corresponding standard fluorophores used for measuring the quantum yields: (top) C6NIB in ethanol (red) and 9,10-DAP in cyclohexane (black); (middle) C6NIB with 100 molar fold TBAH in ethanol (red) and 9,10-DAP in cyclohexane (black); (bottom) C6NIO with 100 molar fold TBAH in ethanol (red) and Rhodamine 6G in ethanol (black).

FIG. 12 shows the fluorescence spectra of C6NIB dispersed in a 1.5×1.5 cm² silica gel TLC plate (containing 0.5 μmol C6NIB and 5 μmol TBAH) recorded at various time intervals after exposure to 1 ppm H₂O₂ vapor. Inset: The emission intensity increase ΔI measured at 553 nm as a function of exposure time, for which the data points are fitted following a first order surface reaction between C6NIB and H₂O₂. The error bars are based on the standard derivations of the intensities as measured.

FIG. 13 shows (a) The fluorescence spectra of C6NIB dispersed in a 1.5×1.5 cm² silica gel TLC plate (containing 0.5 μmol C6NIB and 5 μmol TBAH) measured after 5 min of exposure to various vapor concentrations of H₂O₂, 10, 50, 100, 200, and 1000 ppb. The lowest spectrum (actually two line overlapped each other) was the one measured over the pristine sensor sample and after exposure to the pure water vapor. (b) is a plot showing the emission intensity increase ΔI measured at 553 nm as a function of the vapor concentration of H₂O₂, for which the data points are fitted following the Langmuir adsorption model assuming a quasi-equilibrium reached within 5 min of exposure. The error bars are based on the standard derivations of the intensities as measured.

FIG. 14 shows (a) The fluorescence spectra of C6NIB (5×10⁻⁶ mol/L in ethanol) before addition of TBAH (black, overlapped in the base line spectrum), 5 min (red), 10 min (blue) and 15 min (green) after addition of 5×10⁻⁴ mol/L TBAH (all three spectra overlapped in the base line), and 5 min after addition of 5×10⁻³ mol/L H₂O₂ (magenta); (b) The fluorescence spectra of C6NIB dispersed in a 1.5×1.5 cm² silica gel TLC plate (containing 0.5 μmol C6NIB and 5 μmol TBAH): freshly prepared TLC plate (black), after 5, 10 and 15 min (red, blue, green, all three spectra overlapped in the base line), and after exposure to 225 ppm H₂O₂ vapor for 5 min (magenta).

FIG. 15 shows (a) a time course of the fluorescence intensity change measured at 550 nm for 5×10⁻⁶ mol/L C6NIB ethanol solution after addition of 5×10⁻³ mol/L H₂O₂. Shown in the figure are four series of measurements performed over the sensor solutions containing the same concentrations of C6NIB and H₂O₂, but different concentrations of TBAH, i.e., at molar ratios of TBAH/C6NIB: 1, 10, 100 and 1000. (b) Time course of the fluorescence intensity change measured at 553 nm for C6NIB dispersed in a 1.5×1.5 cm² silica gel TLC plate (containing 0.5 mmol C6NIB) upon exposure to 225 ppm H₂O₂ vapor. Shown in the figure are four series of measurements performed over the TLC plates containing the same molar amount of C6NIB, but different amounts of TBAH, i.e., at molar ratios of TBAH/C6NIB: 1, 10, 50 and 100.

FIG. 16 shows a time course of the fluorescence intensity change measured at 553 nm for C6NIB dispersed in different supporting materials (Al₂O₃ TLC plate, silica gel TLC plate, filter paper), all in the area size of 1.5×1.5 cm², and containing 0.5 μmol C6NIB and 5 μmol TBAH.

Reference will now be made to the exemplary embodiments illustrated, and specific language will be used herein to describe the same. It will nevertheless be understood that no limitation of the scope of the invention is thereby intended.

### DETAILED DESCRIPTION

Reference will now be made to the exemplary embodiments illustrated in the drawings, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

It must be noted that, as used in this specification and the appended claims, the singular forms “a,” “an,” and “the” include plural referents unless the context clearly dictates otherwise. Thus, for example, reference to “a compound” includes one or more of such materials, reference to “an additive” includes reference to one or more of such additives, and reference to “a contacting step” includes reference to one or more of such steps.

### DEFINITIONS

In describing and claiming the present invention, the following terminology will be used in accordance with the definitions set forth below.

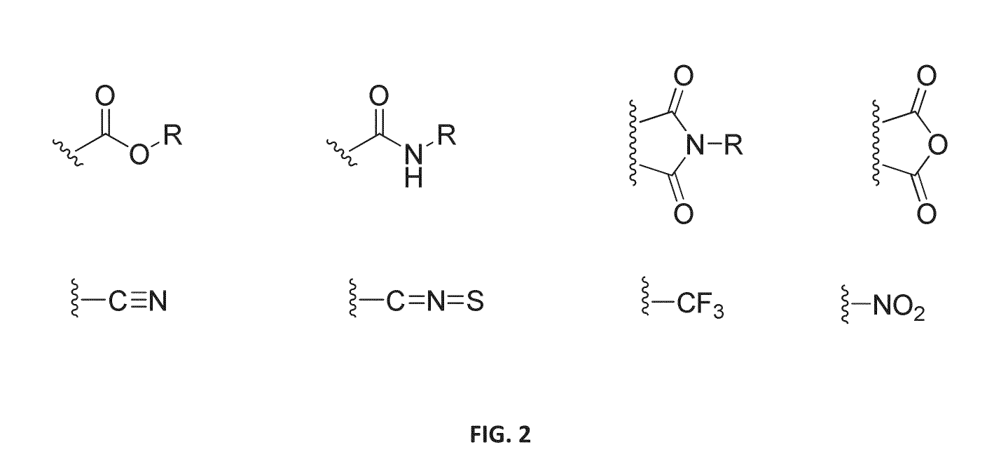

As used herein the phrase “electron withdrawing group” refers to an atom or functional group that removes electron density from a conjugated π system via resonance or inductive electron withdrawal, thus making the π system more electrophilic. Non-limiting examples of electron withdrawing groups that can be used in embodiments of the present invention are shown in FIG. 2.

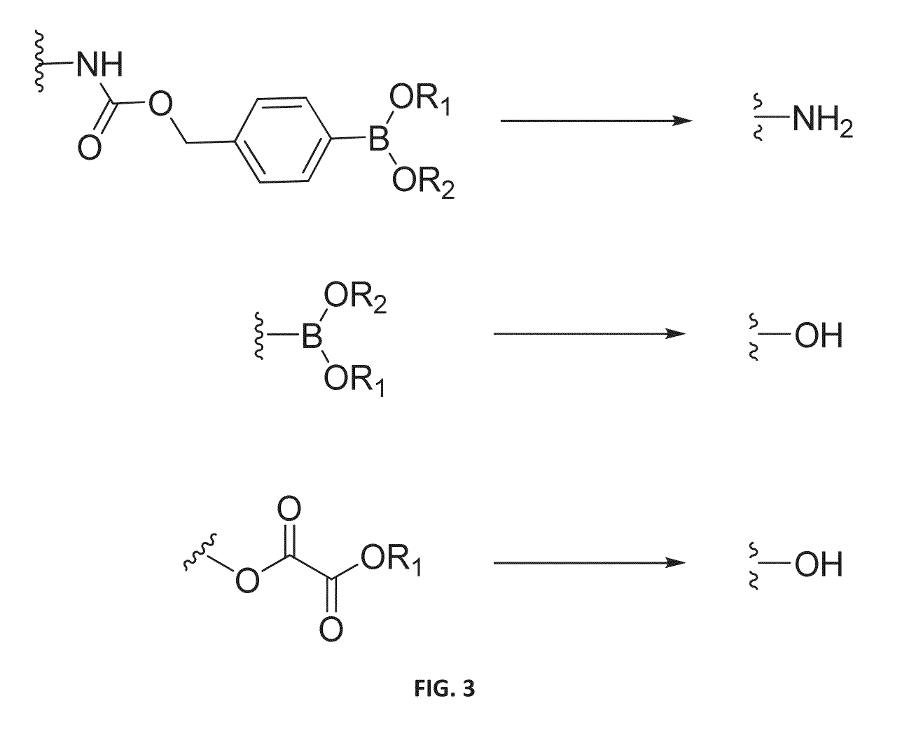

As used herein the phrase “electron donating group” refers to an atom or functional group that donates some of its electron density into a conjugated π system via resonance or inductive electron donation, thereby making the π system more nucleophilic. Non-limiting examples of electron donating groups are shown as the left side components of the reaction schemes shown in in FIG. 3

As used herein the phrase “pre-electron donating group” or “pre-ED” refers to a group that could transition into an electron donating group by reacting with H₂O₂. Non-limiting examples of pre-ED groups are shown in FIG. 3.

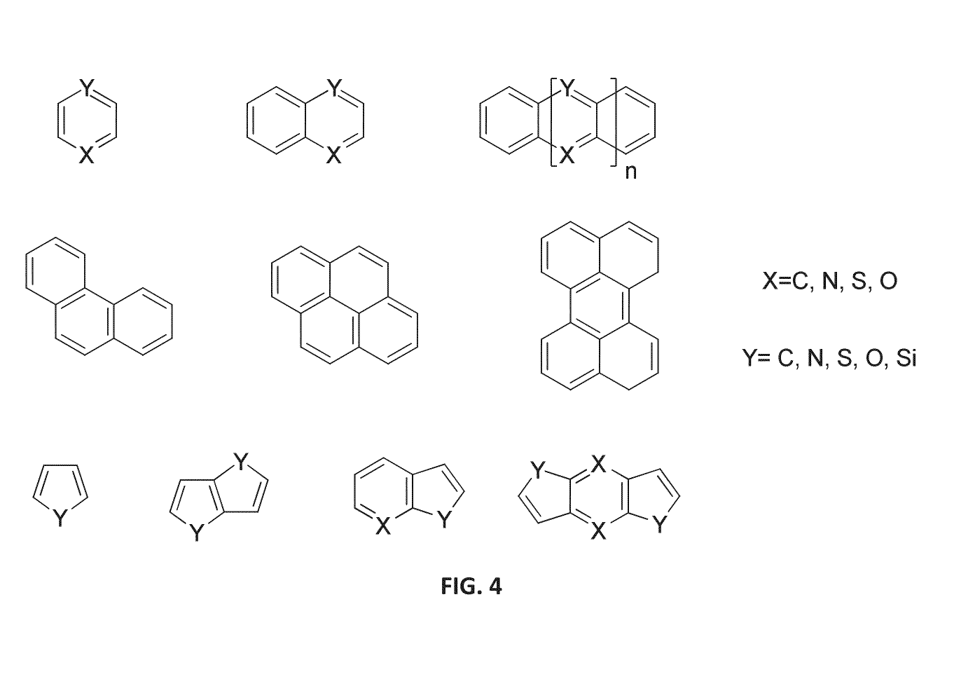

As used herein the phrase “conjugated linker” or “conjugated system” is a system of connected p-orbitals with delocalized electrons in compounds with alternating single and multiple bonds, which in general may lower the overall energy of the molecule and increase its stability. It is noteworthy that lone pairs, radicals or carbenium ions may be part of a conjugated system. Generally, the conjugated system may be cyclic, acyclic, linear or mixed. Non-limiting examples of conjugated systems that may be used in embodiments of the present invention are shown in FIG. 4.

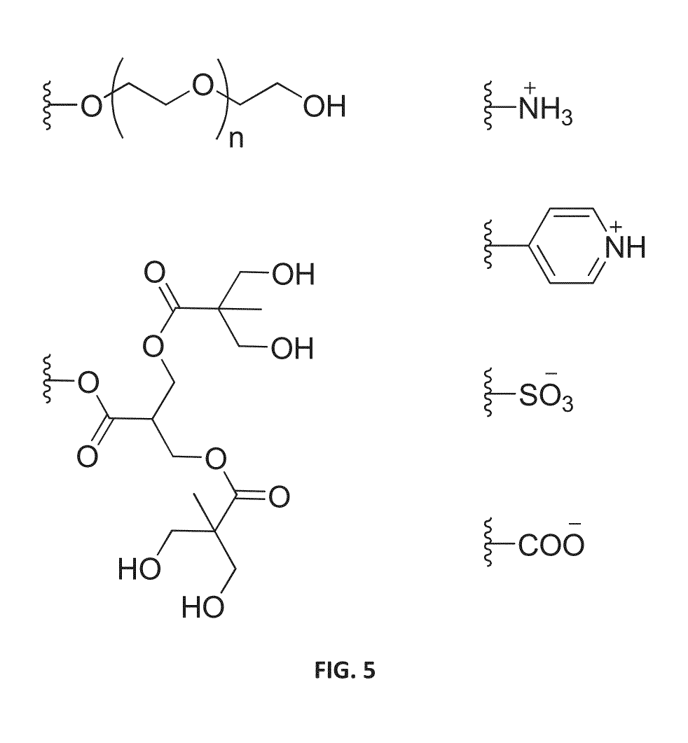

As used herein, the phrase “side chain” refers to a chemical group that can be attached to a conjugated system, an electron withdrawing group or to both. The side can be used to adjust the hydrophobicity of the sensor molecule as a whole. Non-limiting examples of side chains are shown in FIG. 5.

As used herein, the term “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still contain such an item as long as there is no measurable effect thereof.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 10 to about 50” should be interpreted to include not only the explicitly recited values of about 10 to about 50, but also include individual values and sub-ranges within the indicated range. Thus, included in this numerical range are individual values such as 20, 30, and 40 and sub-ranges such as from 10-30, from 20-40, and from 30-50, etc. This same principle applies to ranges reciting only one numerical value. Furthermore, such an interpretation should apply regardless of the breadth of the range or the characteristics being described.

Improvised explosive devices (IEDs), often made of homemade explosives, have raised increasing concern over military defense and national security. TATP (often referred to as the “Mother of Satan”), along with other peroxide explosives including DADP and HMTD, represents one class of the most elusive explosives that can be easily made at home from commercial available products (hydrogen peroxide and acetone). The ease of preparation, together with the tremendous devastative explosive power and easy initiation, makes the peroxide explosives the most preferred by terrorists and insurgents in making IEDs, including suicide bombs. In fact, IEDs (mostly peroxide-based) are one of three major types of explosives required to detect suggested by Department of Homeland Security. However, the detection of these explosives through direct sensing of the peroxide compounds remains difficult mainly due to the weak oxidizing power (weak electron affinity) and lack of nitro-group prevent the normal fluorescence sensing and conventional electronic detection systems, respectively. To this end, hydrogen peroxide, which is often leaked from the organic peroxides as synthetic impurities, is generally considered as a signature compound for detecting the peroxide explosives. Moreover, hydrogen peroxide molecules can also be produced from the chemical decomposition of peroxide explosives, particularly under UV irradiation, which can be set up as part of the sampling system of a device to decompose the peroxide explosive compounds thus collected into gaseous hydrogen peroxide molecules.

Meanwhile, hydrogen peroxide is widely used in industry (waste water processing, paper manufacture) and domestic (bleaching and toothpaste) as well. It also plays a fundamental role in health and disease, for example, the misregulated situation of hydrogen peroxide or other active oxygen species (ROS), may contribute to aging and age-related disease ranging from neurodegeneration to diabetes to cancer. Hence, a single technology that can detect hydrogen peroxide in solution as well as in vapor phase through a fast, economic, and distinctive method is in an urgent demand. However, the current sensing methods can hardly detect hydrogen peroxide vapor efficiently. Even in solution, we still have to face many problems: time consuming synthetic routes, difficulties to modify the structures, and relatively strong fluorescence background (i.e., the pristine state before reacting with hydrogen peroxide is effectively fluorescent).

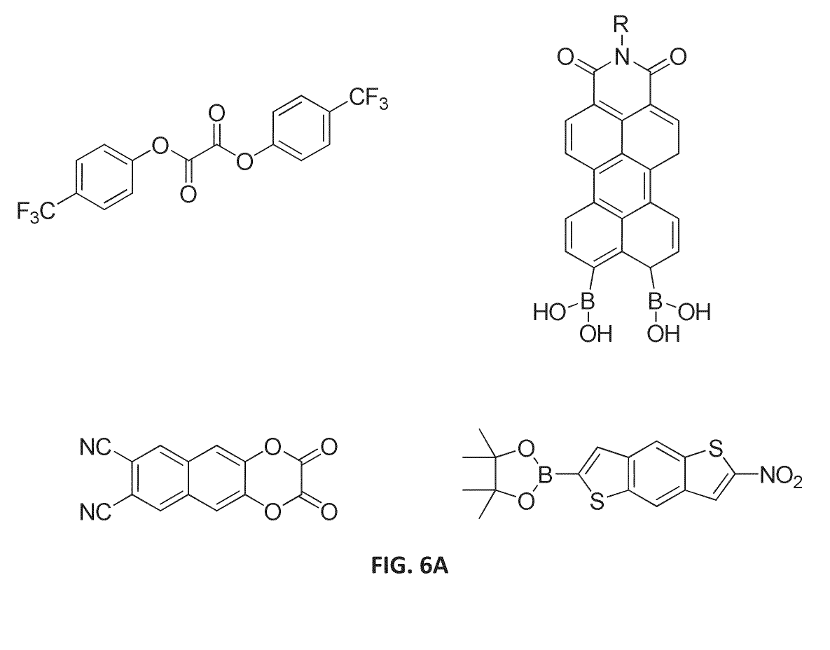

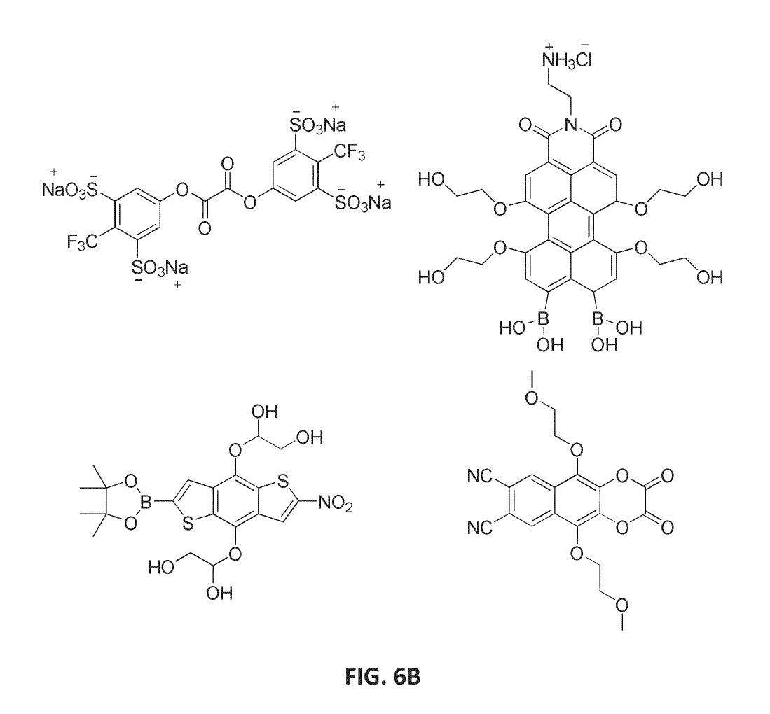

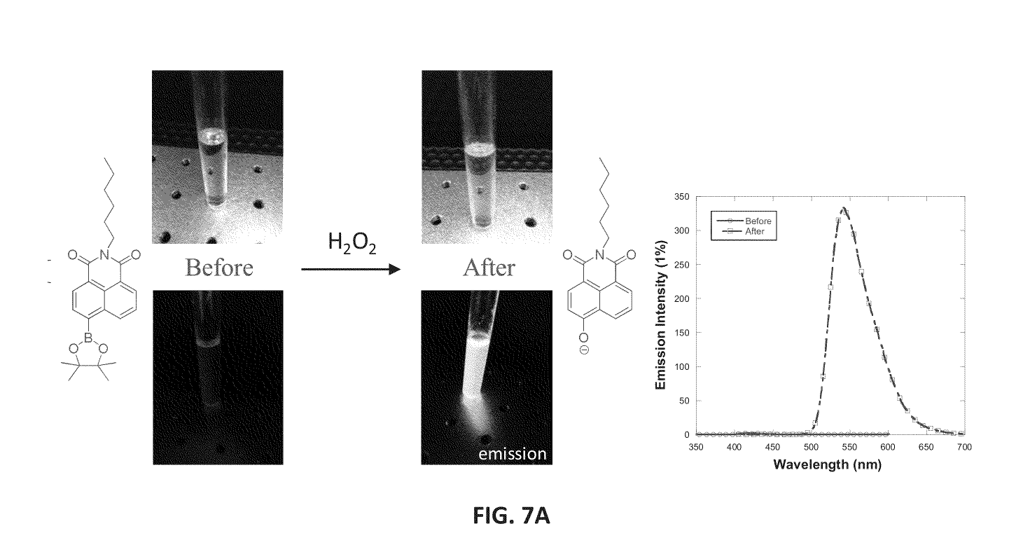

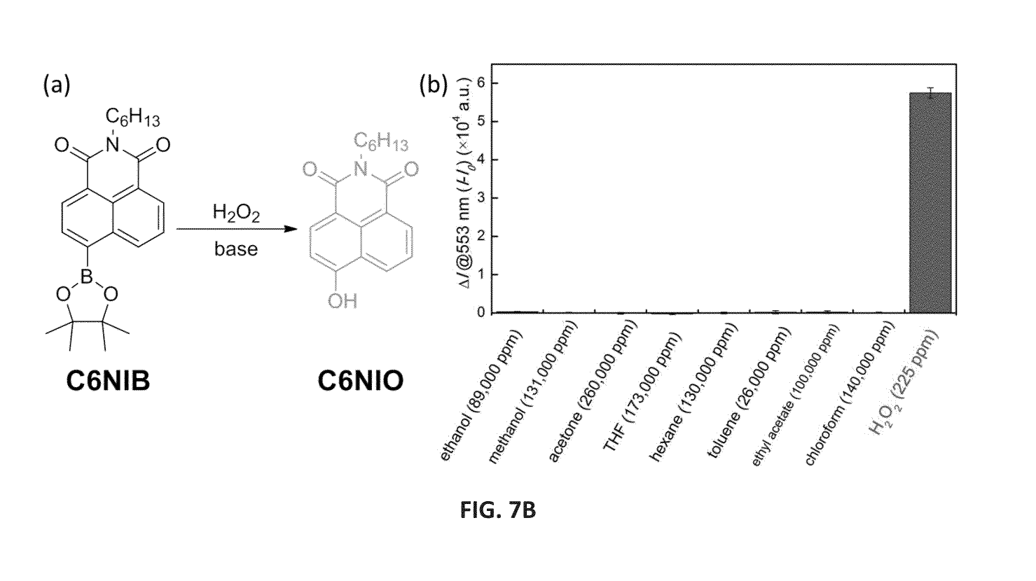

A series of π conjugated molecules, such as those shown in FIGS. 6A, 6B, and 7B capable of detecting hydrogen peroxide both in solution and vapor phase with low detection limit, fast response speed, distinctive selectivity and low cost have been developed by the inventor and are described herein. This series of molecules can possess several features that are ideal for development into sensor system for trace detection of hydrogen peroxide, including, 1) the sensor molecules are easy to make and can function as both colorimetric and fluorescent sensors; 2) the sensor molecules are non-fluorescent or weak fluorescent at pristine state, which means a ‘zero’ background, thus suitable for being used as ‘turn-on’ fluorescence sensors; 3) upon reaction with hydrogen peroxide, the sensor molecule forms an intramolecular charge transfer state by generating a strong O^{δ}-π conjugated electron donor, which can not only increase the molar extinction coefficient in visible light region (leading to colorimetric sensing), but also turn-on the fluorescence (enabling fluorescence sensing); 4) the reaction of sensory molecule with hydrogen peroxide is most favored in pH range 7-8, which is suitable for bio-sensing and living cell imaging; 5) the modification of the side chain (R—) can be readily accomplished, offering large flexibility for adjusting the water solubility of molecules, and the targeting (binding) specificity and other properties relevant to bio-sensing and imaging; 6) upon appropriate modification of the side chain (R—), the sensor molecules can be deposited and bound to 3D porous materials template to enable vapor sensing of hydrogen peroxide, wherein the large surface area and 3D continues porosity are conducive to vapor sampling and detection; while still maintains one-dimensional (1D) stacking and fluorescence properties; 7) upon appropriate modification of the side chain (R—), the sensor molecules can be optimized regarding the size, geometry and solubility that combined will maximize the co-facial π-π stacking between the naphthalimide backbone, leading to 1D self-assemble and thus formation of nanofibril structures, which are ideal materials for vapor sensors because of the large surface area and 3D continues network (once deposited as entangled piling on a substrate).

The π conjugated molecules or compounds that can act to sense the presence of hydrogen peroxide can have the following general structure:

[EW]-[CS]-[PED]

wherein EW is an electron withdrawing group, CS is a conjugated system, and where PED is a pre-electron donating group. The compounds can be configured such that upon exposure to hydrogen peroxide the pre-electron donating group of the π conjugated molecules can react with the hydrogen peroxide to form a π conjugated molecules having a general structure of [EW]-[CS]-[ED], wherein EW is an electron withdrawing group, CS is a conjugated system, and where ED is an electron donating group. Each of EW, CS and ED can include one or more (e.g. up to about five such groups in combination. As noted in each of the above general structures, the π conjugated molecules, both before and after exposure to hydrogen peroxide, can include one or more electron withholding group, one or more conjugated system or linker groups, and one or more pre-electron donating group or electron donating group.

As defined above, the electron withdrawing groups of the π conjugated molecules atoms or functional groups that remove electron density from a conjugated system via resonance or inductive electron withdrawal. Non-limiting examples of electron withdrawing groups that can be used in embodiments of the present invention are shown in FIG. 2. In one embodiment, the electron withdrawing group can be selected from those shown in FIG. 2, or combinations of those structures when more than one electron withdrawing group is present. Electron donating groups are atoms or functional groups that donate some of its electron density into a conjugated system via resonance or inductive electron donation. Non-limiting examples of electron donating groups are shown as the left side components of the reaction schemes shown in in FIG. 3 (right side). Pre-electron donating groups or pre-EDs are groups that can transfer or be modified into an electron donating group by reacting with H₂O₂. Non-limiting examples of pre-ED groups are shown in FIG. 3 (left side). In one embodiment, the pre-ED groups can be selected from those shown on the left of FIG. 3 or combinations thereof. In FIG. 3, R₁ and R₂ can be independently H, alkyl, aryl, or ethylene glycol chain in either liner or branched form.

Conjugated linkers or conjugated systems are system of connected p-orbitals with delocalized electrons in compounds with alternating single and multiple bonds, which in general may lower the overall energy of the molecule and increase its stability. Non-limiting examples of conjugated systems that may be used in embodiments of the present invention are shown in FIG. 4. In one embodiment, the conjugated system can be selected from the general structures shown in FIG. 4 or combinations of those general structures. In one embodiment, the conjugated linkers or conjugated systems can include an aromatic structure. The π conjugated molecules can also include side chains that can be attached to conjugated system(s), the electron withdrawing group(s), or to both. When present, in one embodiment the side chains can act to modify the hydrophilicity of the π conjugated molecule. In one embodiment, the π conjugated molecules include at a side group attached to one or both of the conjugated system and the electron withdrawing group and the side chain is selected from the group of structures shown in FIG. 5.

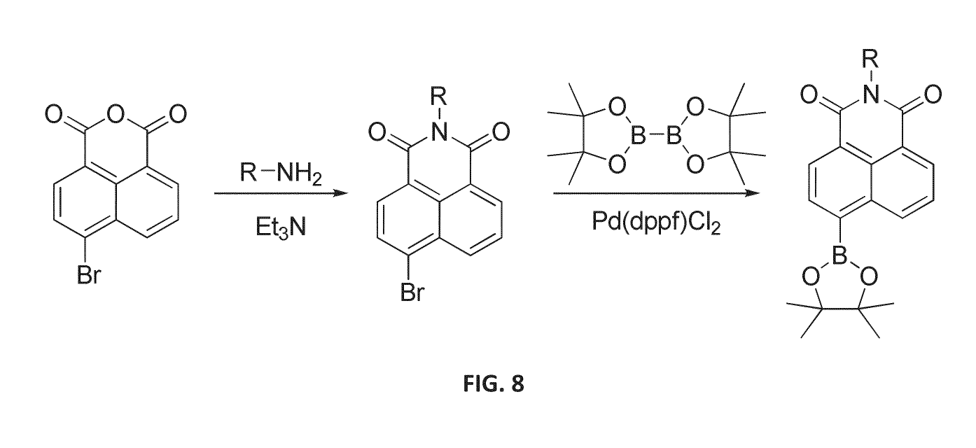

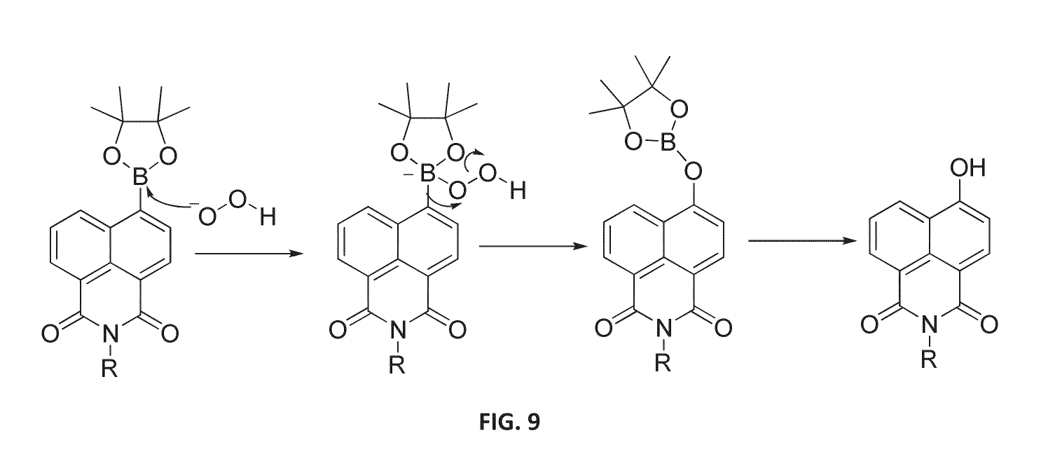

The synthesis of the π conjugated molecules can be fairly straight forward and, based on the teachings herein, readily understood by one of ordinary skill in the art. The synthesis includes taking two steps from the precursor molecules (shown in FIG. 8), which fits the economic and scale up requirement. As the side chains are connected in the conjugation node position, the modification of side chain won't change the electronic properties of the aromatic backbones, offering large flexibility for changing the side-chain so as to optimize the structural and electronic property of the molecule to be suited for sensor application. For example, the absorption spectrum of boron-π conjugated naphthalimide based molecules range in the UV region and its fluorescence efficiency is limited, due to the poor electron transfer process between the imide and boron groups. However, after exposed to hydrogen peroxide and undergoing a rearrangement reaction, the aryl boronate group is turned to an —OH group, which forms a strong O^{δ-} electron donor in basic environment (shown in FIG. 8). The product thus generated possesses much enhanced electron transfer capability from the new formed O^{δ-} moiety (acting as an electron donor) to the imide group. Therefore, stronger absorption in longer wavelength region is observed and the materials are turned to bright yellow color (see FIG. 7A), which is sufficiently visible for “naked-eye” detection. The resulting product also demonstrates strong fluorescence compared to the zero-fluorescence of the pristine sensory material, providing ideal “turn-on” fluorescence sensors with high contrast (signal-to noise ratio) and high sensitivity of signaling. In addition, the reaction between hydrogen peroxide and aryl boronate group (or other Pre-ED group) is specific towards hydrogen peroxide, proving the high selectivity of the sensory molecules. The ease of modification of the side chain can provide facile solubility adjustment, making it feasible to develop water based sensor assay, which can be used in medical and clinical detection of monitoring of hydrogen peroxide. Besides, this series of molecules can also be fabricated into 3D porous sensor materials through both “top-down” and “bottom-up” methods as mentioned above. The porous sensor materials thus fabricated will be suited for vapor sensing of hydrogen peroxide.

For solution based detection of hydrogen peroxide, 2′-7′-dichlorodihydrofluorescein and amplex red/peroxidase systems represent the commonly used methods, though they suffer from nonspecific reactivity with other ROS or require enzymatic additives that are compatible with living cell or live animal specimens. Other aryl boronate based molecules that have been proven workable for fluorescence turn-on sensing of hydrogen peroxide are either hard to modify to enable the water solubility and/or targeting (binding) specificity, or suffer from the significantly high background fluorescence, which results in lower signal-to-noise ratio. Mass spectroscopy (MS), as a usual technique for chemical detection and identification, is highly sensitive, but not suitable for hydrogen peroxide for their reactive and unstable nature.

The new series of molecules disclosed herein provide distinctive selectivity toward hydrogen peroxide due to the reaction nature between aryl boronate group (or other Pre-ED group) and hydrogen peroxide. The low background-fluorescence of the pristine molecules gives a high signal-to-noise ratio and thus achieves significant low detection limit. The response speed of these molecules is also fast because the weak boron-π conjugated bond is facile to break. This series of molecules can be fabricated into 3D porous sensor materials through both “top-down” and “bottom-up” methods as mentioned above. The porous sensor materials thus fabricated are suited for vapor sensing of hydrogen peroxide.

Two major categories of markets considered: military and security, and medical applications. The military and security sectors require non-contact trace detection of explosive particles on a person's body, baggage, vehicle, and cargo. Current technologies cannot detect all required explosives with the speed, specificity, and distance demanded by checkpoint security. Furthermore, current detection systems are expensive and do not always deliver results. Whole Body Imagers (WBIs) used in airports cost $250,000 and have limited efficiency for explosive detections; hand held devices used in the military and police forces cost more than $30,000 dollars per unit. Government officials are becoming reluctant to continue spending money on new devices that fail to deliver the necessary sensitivity.

The sensory materials herein invented can meet the technical requirements for standoff peroxide explosive detection. Current development of vapor sensing of hydrogen peroxide is not as advanced as the solution-based approach. This is mainly because of the lack of appropriate sensory materials that can demonstrate sensitive, selective response to the adsorption of hydrogen peroxide. The molecules disclosed herein can solve these problems, and can provide fast, selective, sensitive and cost-effective sensor systems to detect hydrogen peroxide vapor. These sensor materials can be sold to manufacturers of explosive detectors and other surveillance devices. Such sensors also can be integrated into existing devices and not require their clients, such as the DHS and Military, to replace existing equipment. The materials are suitable for one-time-use with low cost and easy for the periodic replacement.

Medical applications can include imaging. Medical imaging of hydrogen peroxide relies on the turn-on fluorescence, i.e., a fluorescence emission indicates hydrogen peroxide exists in that specific area or domain. An extremely high emission contrast of these sensor materials (close to zero emission background in a pristine state) enables high sensitivity imaging and mapping of hydrogen peroxide in living cells or other physiological media. A weak base condition (pH about 7.4) is usually sufficient for hydrogen peroxide mediated oxidation of a boronate state of the sensor to a phenol state (i.e. to turn on fluorescence). Compared to sensors used for security application, sensors configured for use in medical applications can typically be soluble in water. As a result, the sensor molecules can be functionalized or otherwise includes hydrophilic side chains.

Hydrogen peroxide, along with other reactive oxygen species (ROS), plays a fundamental role in health and disease. For example, the misregulated situation of hydrogen peroxide or other ROS can accumulate and cause oxidative stress inside cells, which may contribute to aging and age-related disease ranging from neurodegeneration to diabetes to cancer. On the other hand, emerging evidences also show that the controlled production of hydrogen peroxide is obligatory to keep cellular fitness. In addition, studies have suggested that hydrogen peroxide and other ROS have critical contribution toward healthy physiological signaling pathways. The new sensor molecules herein can help reveal fundamental new biological insights into the production, localization, trafficking, and in vivo roles of hydrogen peroxide in a wide variety of living systems, including immune, cancer, stem, and neural cell models.

Compared to conventional sensing systems, the present sensors provide a class of simple, expedient technique for vapor detection and analysis. Development of such an efficient sensing technique that can instantly detect and identify hydrogen peroxide vapor will help strengthen the national defense capability (e.g., protecting our soldiers in the battlefields from IED attacks), as well as homeland security. The present sensors can allow nondestructive, standoff detection systems and cheap, disposable detection kits that enable response to the presence of peroxide explosives at a distanced location. Such a response, as indicated by formation of bright color (e.g. yellow), can often be visually identified with a high degree of reliability with unaided visual inspection.

Additionally, the present inventors have recognized that by taking advantage of the high vapor pressure of peroxide compounds, a dual-mode sensor can be designed and operated through vapor sampling of the peroxide explosives.

In one embodiment, the π conjugated molecules disclosed herein can be used to form a sensor for detecting explosives. The sensor can comprise a porous hydrophilic material modified with π conjugated molecules with pre-electron donating group(s), such as an aryl boronate group, the π conjugated molecules having the following general structure:

[EW]-[CS]-[PED]

wherein EW is an electron withdrawing group, CS is a conjugated system, and where PED is a pre-electron donating group. Additionally, the porous hydrophilic material can be capable of detecting hydrogen peroxide vapor by reacting the PED group of the sensor compound with the hydrogen peroxide to provide an optical change, e.g. color or fluorescent change.

While the sensor can be made utilizing a variety of hydrophilic materials known in the art, in one embodiment, the porous hydrophilic material can be a thin film or strip. Generally, the π conjugated molecules disclosed herein can be directly bonded to a hydrophilic material substrate through coating or other methods. A surface coating or binding can be performed through electrostatic interaction or hydrogen bonding between the compound and the surface of the substrate including —OH, —COOH or any other moieties available on the surface. Additionally, direct dispersion of the series of π conjugated molecules solution into a porous matrix (e.g., filter paper or silica gel) can be done to associate the series of π conjugated molecules with a substrate.

The porous films discussed herein can possess at least one of the following: 1) continuous pore channels, allowing for efficient diffusion of the gaseous molecules throughout the film matrix, making it possible to fabricate a thick film to increase the optical density and thus enhance the sensing accuracy; 2) strong hydrophilic (hygroscopic) surface enabling effective adsorption of hydrogen peroxide; 3) nanoscopic structure that allows for maximal distribution of the Pre-ED moiety at the surface, thus enabling maximal exposure to the gaseous analytes; 4) a chemical composition that can effectively stabilize the series of π conjugated molecules 5) weak or no fluorescent property in the pristine state (i.e., before exposure to hydrogen peroxide) allowing for easy monitoring of the fluorescent change. In one embodiment, the porous films discussed herein can possess all of these qualities.

In one embodiment, the porous hydrophilic material can be fabricated by depositing large numbers of nanofibers onto a substrate to form the thin film, where the nanofiber is surface-modified with the π conjugated molecules.

Non-limiting examples of materials that can be used as the porous hydrophilic material include cellulosic paper, filtration paper, silica gel, polymer film, woven polymer, and the like. In one embodiment, the porous hydrophilic material can comprise a cellulose fibril material. Typical hydrophilic or water soluble polymer materials can be commercially available from Dow, including but not limited to, CELLOSIZE® hydroxyethylcellulose (HEC), ETHOCEL® ethylcellulose polymers, KYTAMER® PC polymers, METHOCEL® cellulose ethers, POLYOX® water soluble resins, and the like. In one aspect, a porous silica gel can have greater than 1000 m²/g surface area. In another aspect, these substrates can be substantially transparent. In one embodiment, transparent silica-gel films that possess highly porous structure can be used. The use of such materials can allow for the sensor to be disposable. In one embodiment, the sensor can be disposable.

The colorimetric sensing of hydrogen peroxide is based on highly porous hydrophilic materials modified with π conjugated molecules disclosed herein. Such compounds include one or more aryl boronate group (or other Pre-ED group), which can provide highly selective, strong binding with hydrogen peroxide. For example, the aryl boronate compound is intrinsically colorless (i.e., with no absorption in the visible region), whereas it turns to bright yellow upon reacting the aryl boronate group with hydrogen peroxide forming OH, which then can exhibit strong absorption. The colorimetric change can be used as a visual signal indicative of the presence of hydrogen peroxide as well as with colorimetric detectors, as discussed herein. The emission of the pristine state of this series of molecules will be weak and after exposure to hydrogen peroxide, the fluorescence of the resulting molecules will be enhanced due to the formation of “push-pull” conjugated structure.

As discussed above, the sensors disclosed herein can provide exceptional sensitivity able to detect minute quantities of hydrogen peroxide, in either vapor or liquid form. In one embodiment, the hydrogen peroxide vapor can be less than 1 ppm. In another embodiment, the hydrogen peroxide vapor can be present in an amount of about 1 ppb to about 500 ppb. The sensitivity of the sensor to hydrogen peroxide vapor can be a function of the π conjugated molecules, as well as the concentration of the compound on the hydrophilic material. Although any concentration can be functional, as a general guideline about 50% to about 100% coverage of the surface binding sites can be suitable.

As discussed herein, the present sensors can detect hydrogen peroxide vapors from explosives. As such, in one embodiment, the sensor can detect peroxide based explosives. In another embodiment, the explosives can include a compound selected from the group consisting of triacetone triperoxide (TATP), diacetone diperoxide (DADP), hexamethylene triperoxide diamine (HMTD), and mixtures thereof.

The porous hydrophilic material modified with the series of π conjugated molecules can provide an optical change, such as a color or fluorescent change, upon exposure to the hydrogen peroxide. In this approach, presence of an offending explosive can be qualitatively determined by a perceptible optical change, e.g. change in fluorescence of the material. In one embodiment, the sensor can further include an optical detector, such as a fluorometric detector, associated with the porous hydrophilic material configured to measure the fluorescent change. In this approach, the presence of peroxide can be quantified by correlation with a numeric or other scale. The colorimetric detector can be any suitable detector. Non-limiting examples of suitable detectors can include the commercial photon detector or portable fluorometer.

In one embodiment, the sensor can be disposable (e.g. configured for a single use). In one alternative, the sensor can be a handheld device with the modified hydrophilic material oriented within a receiving port. Thus, the material can be removed and replaced after a predetermined time or when the material otherwise becomes unusable.

A method for detecting explosives or the presence of hydrogen peroxide in a vapor or fluid form is provided. The method can comprise placing a porous hydrophilic material modified with a series of π conjugated molecules in an area having hydrogen peroxide vapor. The π conjugated molecules can have the following structure: [EW]-[CS]-[PED], wherein EW is an electron withdrawing group, CS is a conjugated system, and where PED is a pre-electron donating group. The method can further include identifying an optical change following exposure of the sensor to an area having hydrogen peroxide. In one embodiment, the optical change can be a change in fluorescence. Additionally, the porous hydrophilic material can be capable of detecting of the hydrogen peroxide vapor by reacting the aryl boronate group (or other Pre-ED groups) with the hydrogen peroxide to provide a color change.

In one embodiment, the identifying of the optical change can be accomplished by visual inspection (e.g. a change to yellow emission). In another embodiment, identifying the optical change, e.g. fluorescent change, can be accomplish by use of an optical detector such as a fluorometric detector or other colorimetric device. The measured fluorescent change can be correlated to a numerical or relative scale in order to assist in assessing risk level and/or determining proximity to the explosive. For example, a weak signal may indicate that the tested sample has merely been in recent contact with an explosive while not being a threat in and of itself. A strong signal may indicate an immediate threat and possible actual presence of a corresponding explosive material. Such gradation of signals can help the user to coordinate appropriate response for further investigation and/or management of the risk. Additionally, in one embodiment, the method can further comprise disposing of the porous hydrophilic material after use.

A system for detecting explosives can comprise a porous hydrophilic material modified with a series of π conjugated molecules including any of those described herein and a fluorometric or colorimetric detector associated with the porous hydrophilic material configured to measure the fluorescent or color change. Generally, the porous hydrophilic material can be capable of detecting of the hydrogen peroxide vapor by reacting the aryl boronate group (or other Pre-ED groups) with the hydrogen peroxide to provide a fluorescent or color change. Additionally, the sensor can be a dual mode sensor system. The system can further comprise a UV irradiation source for decomposing peroxide compounds into hydrogen peroxide vapor. As discussed above, two channels of vapor sampling can be employed, one attached with a UV irradiation source, one without. The former can be used for sampling the vapor of peroxide compounds (which can subsequently be decomposed into free hydrogen peroxide molecules by UV irradiation), while the latter can be used for collecting the hydrogen peroxide vapor directly leaked from the raw explosives. The hydrogen peroxide molecules thus collected from both the two channels can be subject to detection by the same colorimetric sensory film placed in the back of the device. With this dual-mode sensing, unprecedented sensitivity can be achieved, as well as increased reliability (to minimize false positives) in detection peroxide explosives, and the related IEDs.

### EXAMPLES

Materials and general instrumentation utilized the examples discussed below are described below. 4-Bromo-1,8-naphthalic anhydride was purchased from TCI America and used as received. PdCl₂(dppf), 1,1″-Bis(diphenylphosphino)ferrocene (dppf) were purchased from Sigma-Aldrich and used as received. Bis(pinacolato)diboron acid, 9,10-diphenylanthracene(9,10-DPA) and Rhodamine 6G were purchased from Fisher Scientific and used as received. Silica gel TLC plates used as supporting matrix for incorporating C6NIB sensor molecules were purchased from EMD Chemicals Inc. (Silicycle Ultrapure Silica Gels SIL-5554-7). For comparison, the filter paper purchased from Whatman (Catalog No. 1001-150) was also used as supporting matrix, but after boiling in deionized water for 1 hour to remove the bleaching reagents contained in the paper. All organic solvents were purchased from commercial manufacturers and used as received.

UV-vis absorption spectra were measured on a PerkinElmer Lambda 25 spectrophotometer or Agilent Cary 100. The fluorescence spectra were measured on a PerkinElmer LS 55 spectrophotometer or Agilent Eclipse spectrophotometer. The fluorescence spectra of solid sample (e.g., TLC plates) were recorded on an Ocean Optics USB4000 equipped with 395 nm LED light source and optical fiber (Avantes, FCR-UV200/600-2-IND) for light delivery. ¹H and ¹³C NMR spectra were recorded on a Varian Unity 300 MHz Spectrometer at room temperature in appropriate deuterated solvents. All chemical shifts are reported in parts per million (ppm). ESI MS spectra were recorded on a Micromass Quattro II Triple Quadrupole Mass Spectrometer, and the solvent used was methanol.

### Example 1

### Synthesis of Exemplary Sensor Molecule C6NIB

The synthesis route of sensor molecule C6NIB is described below and shown generally in FIG. 8. The synthesis occurs begins when 6-bromo-2-hexyl-1H-benzo[de]isoquinoline-1,3(2H)-dione: 4-Bromo-1,8-naphthalic anhydride (1 g, 3.6 mmol), hexylamine (383 mg, 3.8 mmol), triethylamine (10 mL) were added into 50 mL anhydrous ethanol and refluxed for 4 hours. The reaction mixture was evaporated under reduced pressure and then purified through column chromatography on silica gel with hexane/ethyl acetate (5:1, v/v) as eluent. The product was obtained as white crystal (1.10 g, 85%). ¹H NMR (CDCl₃, 300 MHz, ppm): δ=8.52-8.55 (1H, m, Ar—H), 8.39-8.43 (1H, m, Ar—H), 8.27-8.29 (1H, d, J=7.8 Hz, Ar—H), 7.90-7.93 (1H, d, J=7.8 Hz, Ar—H), 7.71-7.76 (1H, m, Ar—H), 4.07-4.13 (2H, t, J=7.2 Hz, CH₂), 1.66-1.71 (2H, m, CH₂), 1.29-1.40 (6H, m, CH₂), 0.84-0.8 (3H, t, CH₃). ¹³C NMR (CDCl₃, 75 MHz, ppm): δ 163.32, 163.29, 132.90, 131.76, 130.94, 130.88, 129.95, 128.65, 127.87, 122.90, 122.05, 40.51, 31.45, 27.91, 26.70, 22.49, 14.00.

2-hexyl-6-(4,4,5,5-tetramethyl-1,3,2-dioxaborolan-2-yl)-1H-benzo[de]isoquinoline-1,3(2H)-dione: 6-bromo-2-hexyl-1H-benzo[de]isoquinoline-1,3(2H)-dione (360 mg, 1 mmol), anhydrous potassium acetate (588 mg, 6 mmol), bis(pinacolato)diboron (560 mg, 2.2 mmol), [PdCl₂ (dppf)] (73 mg, 10 mol %), and dppf (55 mg, 10 mol %) and 20 mL DMF were mixed and degassed by three freeze-pump-thaw cycles. The reaction mixture was heated at 120° C. for 3 hours. After cooling to room temperature, the reaction mixture was partitioned between water and dichloromethane. The aqueous phase was extracted with 20 mL dichloromethane for 3 times and then combined with the original dichloromethane phase. This dichloromethane solution was washed with brine twice and then washed with water, followed by drying with Na₂SO₄. After rotary evaporate under reduced pressure to remove excess solvent, the residue was purified through column chromatography on silica gel with hexane/ethyl acetate (5:1, v/v) as eluent. The product was obtained as white powder (180 mg, 44%). ¹H NMR (CDCl₃, 300 MHz, ppm): δ=9.05-9.08 (1H, m, Ar—H), 8.50-8.56 (2H, m, Ar—H), 8.24-8.26 (2H, d, J=7.2 Hz, Ar—H), 7.71-7.76 (1H, t, J=7.2 Hz, Ar—H), 4.11-4.16 (2H, t, J=7.2 Hz, CH₂), 1.71 (2H, m, CH₂), 1.30-1.35 (6H, m, CH₂), 0.85-0.90 (3H, t, CH₃). ¹³C NMR (CDCl₃, 75 MHz, ppm): δ 164.16, 164.14, 135.66, 135.11, 134.77, 130.70, 129.59, 127.69, 126.95, 124.62, 122.51, 84.48, 40.43, 31.50, 27.96, 26.74, 24.91, 22.50. ESI-LRMS m/z: Calcd for C₂₄H₃₀BNO₄: 407.2. Found: 408.3 [M+H]⁺.

### Example 2

### Dispersion of Sensor Molecules in Silica Gel TLC Plate and Filter Paper Matrix

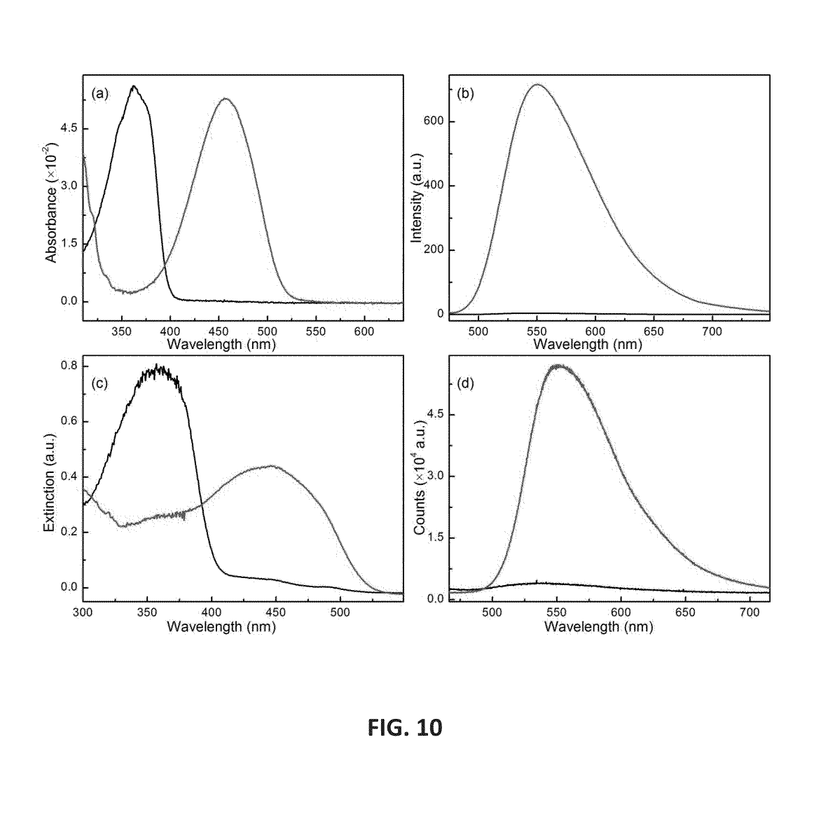

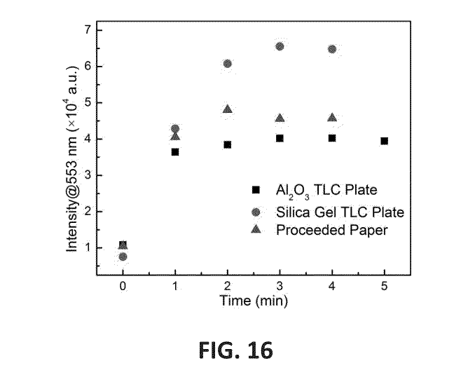

50 μL ethanol solution of C6NIB at different concentrations (also containing appropriate concentrations of TBAH as detailed below) were drop-cast uniformly onto a 1.5×1.5 cm² silica gel TLC plate, followed by drying at room temperature in vacuum for 1 hour. To adjust the molar amount of C6NIB loading (as indicated in FIG. 10), various concentrations of C6NIB in ethanol were prepared and used: 0.1, 0.01 and 0.001 mol/L. Uniform dispersion of C6NIB sensor molecules within the TLC plate is indicated by the uniform emission density shown in the emission photography of the plate after exposure to the H₂O₂ vapor (not shown). The same dispersion method was also used for dispersing the sensor molecules into Al₂O₃ TLC plate and filter paper, which were used for comparative sensor investigation as shown in FIG. 16.

### Example 3

### Absorption (Extinction) Spectral Measurement

Due to the non-transparency of the TLC plate, the absorption spectra of the sensors dispersed in this medium had to be measured in reflection mode, which can then be converted into extinction spectral data (in analogy to light absorption). The reflection spectra were recorded on a PerkinElmer Lambda 650R spectrophotometer with a build-in universal reflection accessory. The spectra were collected with unpolarized light incident at ˜45° with respect to the surface and integrated for 0.1 s and at a resolution of 1 nm. The spectra collected were converted and shown as extinction measured as −log(R/R₀), where R is the reflectance of the loaded sample substrate and R₀ is the reflectance of the unload TLC plate substrate.

### Example 4

### Flourescence Quantum Yield Measurement

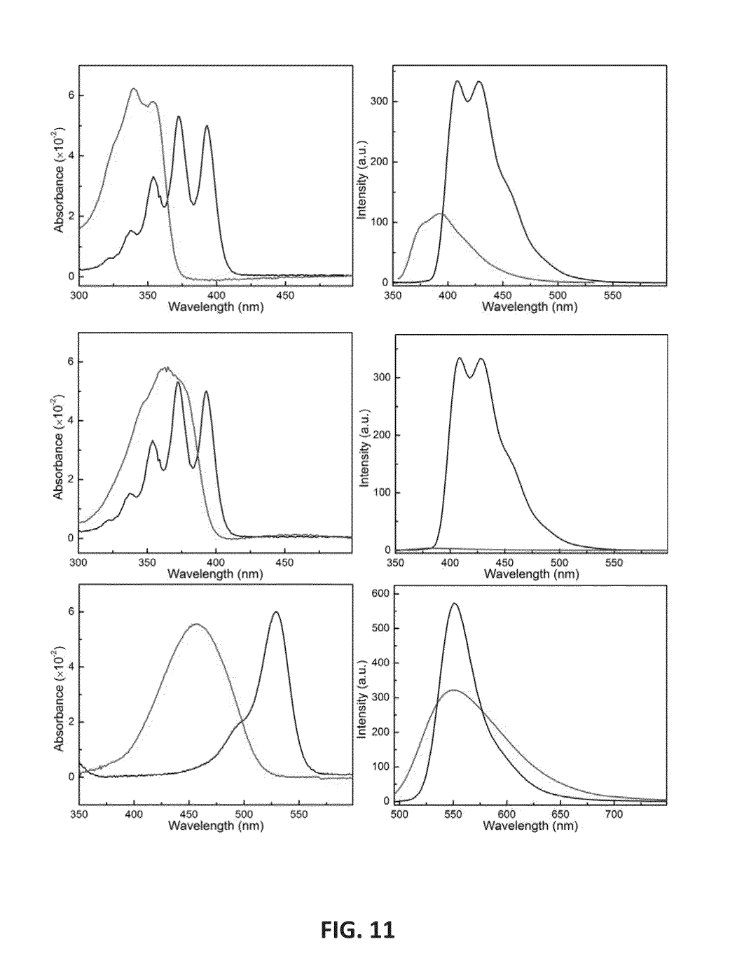

As shown in FIG. 11, molecule C6NIB is only weakly fluorescent in the UV region (λₘₐₓ at 392 nm) mainly due to the π-π* transition of naphthalimide backbone, while C6NIO is strongly fluorescent in the longer wavelength region (λₘₐₓ at 550 nm) due to the charge transfer transition. The quantum yield (Φ) of C6NIB and C6NIO were determined by a single-point measurement with standard sample of known quantum yield. 9,10-diphenylanthracene(9,10-DPA) (Φ=0.95 in cyclohexane) and Rhodamine 6G (Φ=0.94 in ethanol)⁶ were chosen as standard samples for C6NIB and C6NIO, respectively. The excitation wavelengths were selected as 340 nm and 480 nm for 9,10-DPA/C6NIB and Rhodamine 6G/C6NIO, respectively. The quantum yields of C6NIB and C6NIO in ethanol (in the presence of 100 fold TBAH) were determined as 0.069 and 0.254, respectively. The quantum yield of C6NIB in ethanol (in the presence of 100 fold TBAH) is only 0.006.

### Example 5

### Sensor Stability in Solution and Silica Gel TLC Plate

Fluorescence spectra of 3 mL of 5×10⁻⁶ mol/L C6NIB ethanol solution were recorded before and after addition of 5×10⁻⁶ mol/L TBAH (FIG. 11 top left). Spectra were also recorded at different time intervals after addition of TBAH. After addition of 5×10⁻³ mol/L H₂O₂, the fluorescence emission was tuned on immediately due to the quick reaction, and fluorescence spectrum was recorded 5 min after addition of H₂O₂. The C6NIB dispersed TLC plate sample was prepared according to the method description above, and fluorescence spectra were measured at different time intervals after preparation (FIG. 11 top right), which did not show significant change in fluorescence spectra or intensity within the experimental investigation period. The same TLC plate was then exposed to 225 ppm H₂O₂ vapor for 5 minutes, followed by measurement of the fluorescence spectrum.

### Example 6

### Time Course of Sensor Response in Solution and Solid Matrices

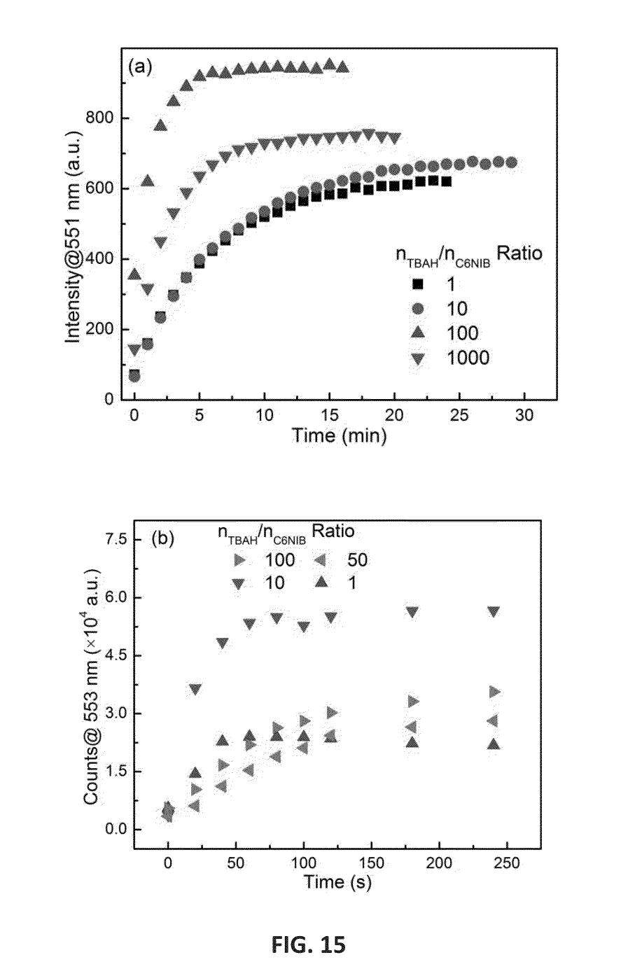

To find the optimal concentration of TBAH (or molar ratio TBAH/C6NIB) that would give the fasted sensor reaction, i.e., the H₂O₂ mediated oxidation of C6NIB to C6NIO (as shown in FIG. 7B), we measured the time course of the fluorescence intensity change at 550 nm for a 3 mL 5×10⁻⁶ mol/L C6NIB ethanol solution after addition of 5×10⁻³ mol/L H₂O₂ (FIG. 15a), which was measured under four different concentrations of TBAH, i.e., at molar ratios of TBAH/C6NIB: 1, 10, 100 and 1000. Apparently, the solution containing TBAH at a molar ratio of TBAH/C6NIB of 100 demonstrated the fasted sensor response.

The similar optimization experiments were performed for the sensor molecules dispersed in silica gel TLC plates as shown in FIG. 15b, where the time course of the fluorescence intensity change was measured at 553 nm for C6NIB dispersed in a 1.5×1.5 cm² silica gel TLC plate (containing 0.5 μmol C6NIB) upon exposure to H₂O₂ vapor fixed at 225 ppm. Four series of measurements were performed over the TLC plates containing the same molar amount of C6NIB, but different amounts of TBAH, i.e., at molar ratios of TBAH/C6NIB: 1, 10, 50 and 100. The testing experiment was performed by hanging the loaded TLC plate in the saturated vapor of H₂O₂ (225 ppm) above 10 mL of 35 wt % H₂O₂ solution sealed in a 50 mL jar. The fluorescence emission evolved at different time intervals was measured by Ocean Optics USB4000 spectrophotometer.

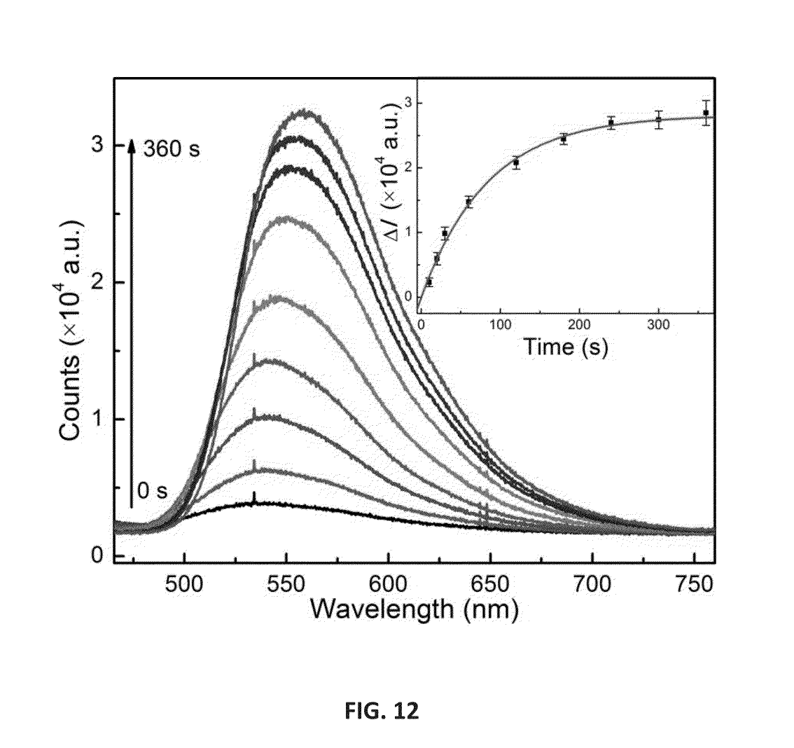

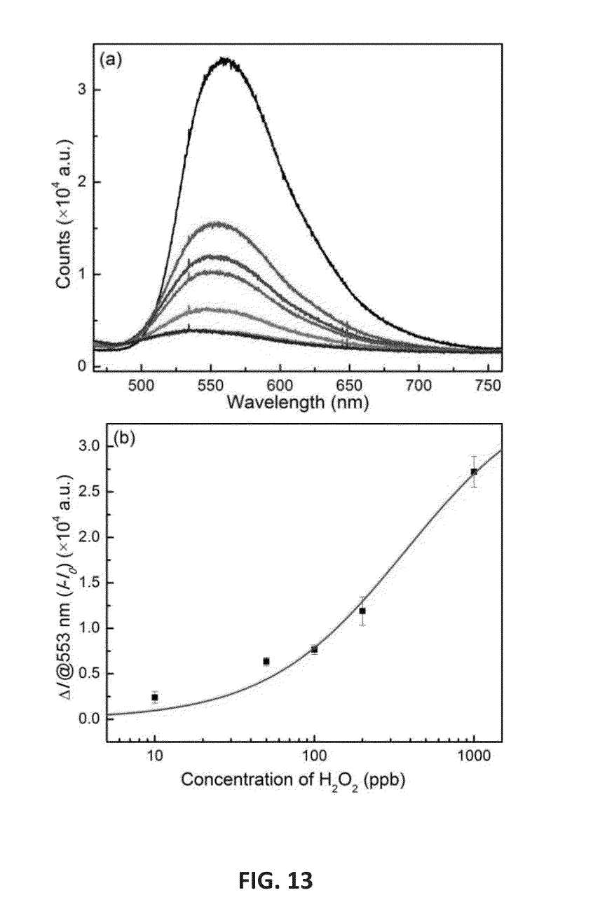

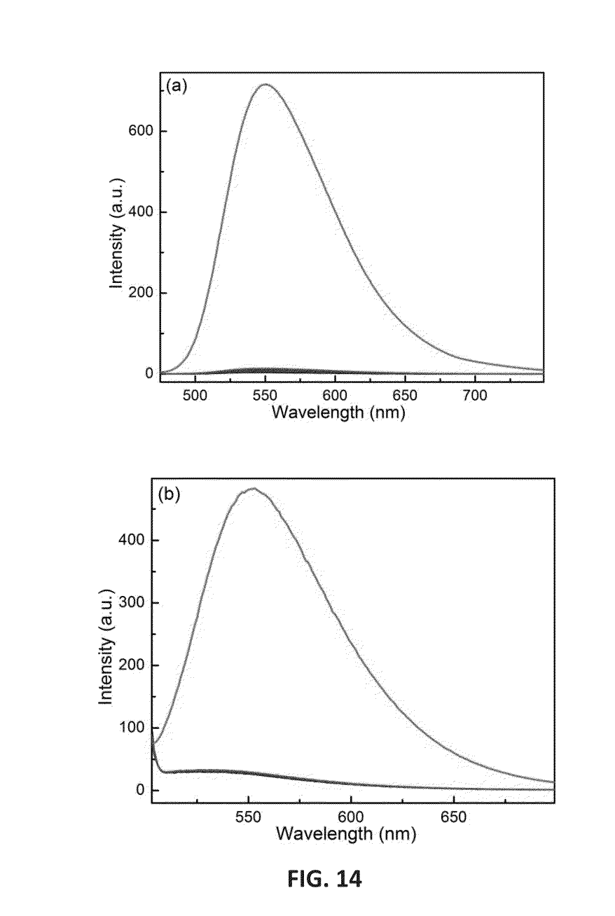

For the measurements performed under varying vapor concentrations of H₂O₂ (shown FIGS. 10 and 12), the testing experiment was performed by hanging the loaded TLC plate in the saturated vapor of H₂O₂ generated in a 26.5 L container, where approximately 1 L of H₂O₂ solution (diluted down to various concentrations) was put in a and sealed for 12 hours to reach the equilibrium vapor pressure. The equilibrium vapor pressure corresponding to a specific diluted concentration of H₂O₂ solution was deduced from the literature. In the container, continuous vapor stream was produced by a mini fan (Radio Shack, 40 mm, 12VDC, 6500 RPM), and the sensor loaded TLC plate was placed against the vapor stream (distanced from the fan by 0.5 cm), and about 20 cm above the solution surface. After exposure to the vapor for different time intervals, the TLC plate was taken out for fluorescence measurement. In this study, various diluted concentrations of H₂O₂ solution were obtained by diluting the commercial 35 wt % solution with pure water 100, 500, 1000, 2000, and 10000 times, which produce saturated (equilibrium) vapor pressures of H₂O₂ of 1000, 200, 100, 50 and 10 ppb, respectively.

### Example 7

### Comparison of Sensor Response Between Different Supporting Matrices

The sensor testing as shown in FIG. 15b was also performed over the sensor molecules dispersed in alumina TLC plate and filter paper. Although these two materials also possess large porosity and surface area, the sensor efficiency (regarding both response speed and fluorescence turn-on ratio at saturate stage) was found significantly lower than that observed for silica gel TLC plate. This is likely due to the hydrophilic surface of silica gel that is more conducive for homogeneous dispersion of TBAH/C6NIB as discussed in the main context of this manuscript.

### Example 7

### Selectivity Test

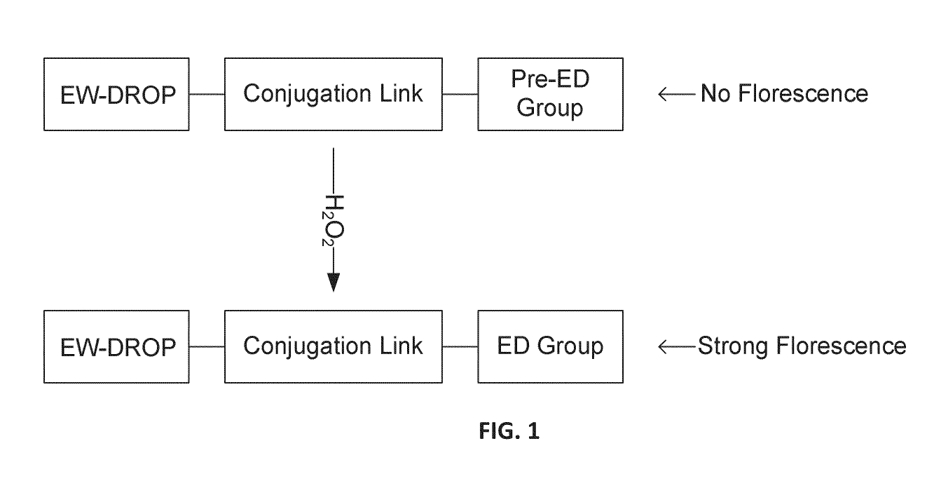

The sensor loaded TLC plate tested in FIG. 1 15 and 16 were exposed to the saturated vapor of various common solvents such as ethanol (89,000 ppm), methanol (131,000 ppm), acetone (260,000 ppm), THF (173,000 ppm), hexane (130,000 ppm), toluene (26,000 ppm), ethyl acetate (100,000 ppm), chloroform (140,000 ppm), as shown in FIG. 7B, to validate the selectivity of the sensor molecule. The increase in fluorescence intensity was measured at 553 nm over C6NIB loaded silica gel TLC plate (the same component as used in FIG. 16) after 5 min exposure to 225 ppm H₂O₂ vapor, in comparison to that upon exposure to the saturated vapor of the common solvents. Although the vapor pressures of the reference solvents are about three orders of magnitude higher than that of H₂O₂, our experiments did not demonstrate any significant fluorescence emission evolution even after extensive exposure to these highly concentrated solvents vapor. This clearly proves the high selectivity of the sensor molecule C6NIB for detection of H₂O₂.

It is to be understood that the above-referenced arrangements are only illustrative of the application for the principles of the present invention. Numerous modifications and alternative arrangements can be devised without departing from the spirit and scope of the present invention. While the present invention has been shown in the drawings and fully described above with particularity and detail in connection with what is presently deemed to be the most practical and preferred embodiment(s) of the invention, it will be apparent to those of ordinary skill in the art that numerous modifications can be made without departing from the principles and concepts of the invention as set forth herein.

What is claimed is:

1. A sensor for detecting hydrogen peroxide, comprising
a porous hydrophilic material modified with π conjugated molecules with the following general structure:
[EW]-[CS]-[PED]
wherein EW is an electron withdrawing group, CS is a conjugated system, and where PED is a pre-electron donating group; and
wherein the porous hydrophilic material is capable of detecting hydrogen peroxide upon reaction of the PED group with the hydrogen peroxide to provide an optical change.

2. A sensor of claim 1, wherein upon exposure to hydrogen peroxide the pre-electron donating group of the π conjugated molecules reacts with the hydrogen peroxide to form a π conjugated molecule having a general structure of: EW-CS-ED, wherein EW is an electron withdrawing group, CS is a conjugated system, and where ED is an electron donating group.

3. The sensor of claim 1, wherein the porous hydrophilic material is a thin film.

4. The sensor of claim 1, wherein the porous hydrophilic material comprises a cellulose fibril material.

5. The sensor of claim 1, wherein the porous hydrophilic material is a nanofiber surface-modified with a series of π conjugated molecules wherein the conjugated system of the π conjugated molecules is selected from the group consisting of: 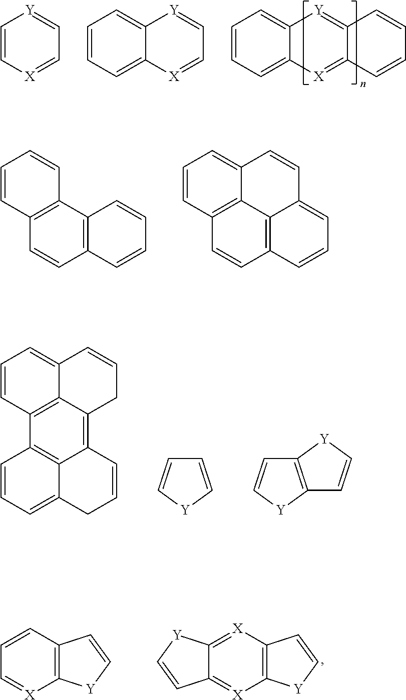
and combinations thereof, where X is C, N, S or O, Y is C, N, S, O or Si and n is a positive integer less than 7.

6. The sensor of claim 1, wherein the electron withdrawing group of the π conjugated molecules has a structure selected from the group consisting of: 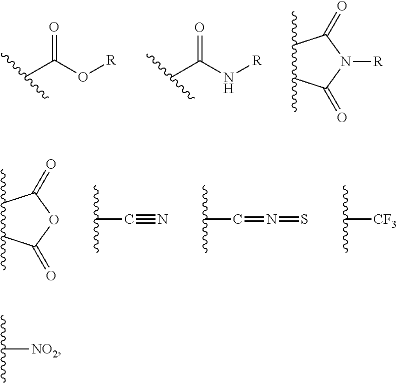
and combinations thereof.

7. The sensor of claim 1, wherein the pre-electron donating group of the π conjugated molecules has a general structure selected from the group consisting of 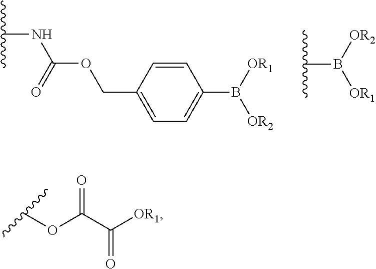
and combinations thereof, wherein R₁ is H, alkyl, aryl or ethylene glycol chain in either liner or branched form, and R₂ is H, alkyl, aryl or ethylene glycol chain in either liner or branched form.

8. The sensor of claim 1, wherein the conjugated system, the electron withdrawing group, or both has a side chain attached thereto.

9. The sensor of claim 9, wherein the side chain modifies the hydrophilicity of the π conjugated molecule.

10. The sensor of claim 9, wherein the side chain has a structure selected from the group consisting of: 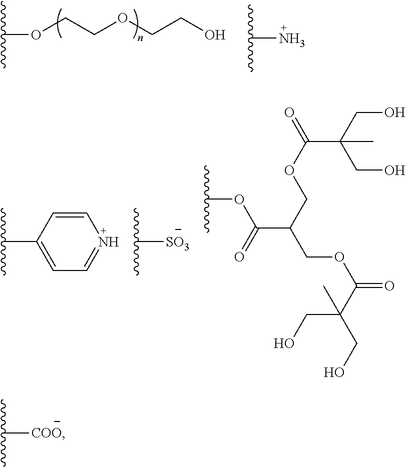
and combinations thereof.

11. The sensor of claim 1, wherein conjugated link is an aromatic compound.

12. The sensor of claim 1, wherein the sensor can detect hydrogen peroxide present in amounts less than 1 ppm.

13. The sensor of claim 1, wherein the sensor can detect hydrogen peroxide present in an amount of about 1 ppb to about 500 ppb.

14. The sensor of claim 1, wherein the sensor can detect peroxide based explosives.

15. The sensor of claim 14, wherein the peroxide based explosive can include a compound selected from the group consisting of triacetone triperoxide, diacetone diperoxide, hexamethylene triperoxide diamine, and mixtures thereof.

16. The sensor of claim 1, wherein the porous hydrophilic material modified with a series of π conjugated molecules provide fluorescent change upon exposure to the hydrogen peroxide.

17. The sensor of claim 1, wherein the optical change upon exposure is fluorescence or color.

18. The sensor of claim 1, further comprising an optical detector associated with the porous hydrophilic material configured to measure the optical change.

19. The sensor of claim 18, wherein the optical detector is a fluorimetric detector.

20. The sensor of claim 1, wherein the sensor is disposable.

21. A method for detecting explosives, comprising
placing a porous hydrophilic material modified with a series of π conjugated molecules in an area having hydrogen peroxide and assessing an optical change of the porous hydrophilic material modified with a series of π conjugated molecules;
wherein the series of π conjugated molecules having the following structure:
[EW]-[CS]-[PED], and
wherein EW is an electron withdrawing group, CS is a conjugated system, and where PED is a pre-electron donating group; and
wherein the porous hydrophilic material is capable of detecting hydrogen peroxide upon reaction of the PED group with the hydrogen peroxide to provide an optical change.

22. The method of claim 21, wherein assessing of the optical change is by visual inspection.

23. The method of claim 21, wherein the assessing of the optical change is by a fluorescent or colorimetric device.

24. The method of claim 21, wherein the method provides for detection of hydrogen peroxide present in an amount of less than 1 ppm.

25. The method of claim 21, wherein the method provides for detection of hydrogen peroxide present in an amount of about 1 ppb to about 500 ppb.

26. The method of claim 21, further comprising disposing the porous hydrophilic material after use.
